(19) United States
(12) Patent Application Publication
(10) Pub. No.: US 2006/0019273 A1
(43) Pub. Date: Jan. 26, 2006

(54) Detection card for analyzing a sample for a target nucleic acid molecule, and uses thereof

(76) Inventors: Dennis M. Connolly, Rochester, NY (US); Jeffrey Hainon, Webster, NY (US); Richard S. Murante, Rochester, NY (US); Roberta J. Greco, Canandaigua, NY (US); Craig W. Tiller, Rochester, NY (US)

Correspondence Address: Integrated Nano-Technologies, LLC, 999 Lehigh Station Road, Henrietta, NY, 14467, US

(21) Appl. No.: 11/128,079

(22) Filed: May 12, 2005

Related U.S. Application Data
(60) Provisional application No. 60/570,331, filed on May 12, 2004.

(51) Int. Cl. C12Q 1/68; C12M 1/34

(52) U.S. Cl. 435/6; 435/287.2

(57) ABSTRACT

The present invention relates to a detection card made of a first planar element having a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card also has a second planar element parallel to and joined with the first planar element along a first planar interface. A fluid pathway is formed proximate to the first planar interface and includes a detection reservoir into which at least part of the detection chip is received. Also disclosed is a system and a method for detecting a target molecule, both of which utilize the detection card.

This application claims the benefit of U.S. Provisional Patent Application Serial No. 60/570,33 1, filed May 12, 2004, which is hereby incorporated by reference in its entirety.

### FIELD OF THE INVENTION

This invention relates to a detection card for analyzing a sample for a target nucleic acid molecule, and systems and methods for detecting a target nucleic acid molecule in a sample using the detection card.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification, and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics 4:560 (1989) and the ligase chain reaction described in European Patent No. 0320308B 1. Transcription-based amplification methods are described in detail in U.S. Pat. Nos. 5,766,849 and 5,654,142, Kwoh et al., Proc. Natl. Acad. Sci. U.S.A. 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol. 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve 21(8):1064 (1998); Wiedbrauk et al., Journal of Clinical Microbiology 33(10):2643-6 (1995); Deneer and Knight, Clinical Chemistry 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology 39(2):485-493 (2001); Al-Soud et al., Journal of Clinical Microbiology 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092, and PCT WO 90/15070. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection of the presence of specific DNA sequences. Most technologies related to array chips involve the coupling of a probe of known sequence to a substrate that can either be structural or conductive in nature. Structural types of array chips usually involve providing a platform where probe molecules can be constructed base by base or by covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location. Alternatively, conductive types of array chips contain probe sequences linked to conductive materials such as metals. Hybridization of a target nucleic acid typically elicits an electrical signal that is carried to the conductive electrode and then analyzed.

For most solid support or array technologies, small oligonucleotide capture probes are immobilized or synthesized on the support. The sequence of the capture probes imparts the specificity for the hybridization reaction. Several different chemical compositions exist currently for capture probe studies. The standard for many years has been straight deoxyribonucleic acids. The advantage of these short single stranded DNA molecules is that the technology has existed for many years and the synthesis reaction is relatively inexpensive. Furthermore, a large body of technical studies is available for quick reference for a variety of scientific techniques, including hybridization. However, many different types of DNA analogs are now being synthesized commercially that have advantages over DNA oligonucleotides for hybridization. Some of these include PNA (protein nucleic acid), LNA (locked nucleic acid) and methyl phosphonate chemistries. In general, all of the DNA analogs have higher melting temperatures than standard DNA oligonucleotides and can more easily distinguish between a fully complementary and single base mis-match target. This is possible because the DNA analogs do not have a negatively charged backbone, as is the case with standard DNA. This allows for the incoming strand of target DNA to bind tighter to the DNA analog because only one strand is negatively charged. The most studied of these analogs for hybridization techniques is the PNA analog, which is composed of a protein backbone with substituted nucleobases for the amino acid side chains (see www.appliedbiosystems.com or www.eurogentec.com). Indeed, PNAs have been used in place of standard DNA for almost all molecular biology techniques including DNA sequencing (Arlinghaus et al., Anal Chem. 69:3747-53 (1997)), DNA fingerprinting (Guerasimova et al., Biotechniques 31:490-495 (2001)), diagnostic biochips (Prix et al., Clin. Chem. 48:428-35 (2002); Feriotto et al., Lab Invest 81:1415-1427 (2001)), and hybridization based microarray analysis (Weiler et al., Nucleic Acids Res 25:2792-2799 (1997); Igloi, Genomics 74:402-407 (2001)).

Techniques for forming sequences on a substrate are known. For example, the sequences may be formed according to the techniques disclosed in U.S. Pat. No. 5,143,854 to Pirrung et al., PCT Publication No. WO 92/10092, or U.S. Pat. No. 5,571,639 to Hubbell et al. Although there are several references on the attachment of biologically useful molecules to electrically insulating surfaces such as glass (http://www.piercenet.com/Technical/default.cftn?tmpl=./Lib/ViewDoc.cfm& doc=3483; McGovern et al., Langmuir 10:3607-3614 (1994)) or silicon oxide (Examples 4-6 of U.S. Pat. No. 6,159,695 to McGovern et al.), there are few examples of effective molecular attachment to electrically conducting surfaces except for gold (Bain et al., Langmuir 5:723-727 (1989)) and silver (Xia et al., Langmuir 22:269, (1998)). In general, the problem of attaching biologically active molecules to the surface of a substrate, whether it is a metal electrical conductor or an electrical insulator such as glass, is more difficult than the simple chemical reaction of a reactive group on the biological molecule with a complementary reactive group on the substrate. For example, a metal electrical conductor has no reactive sites, in principle, except those that may be adventitiously or deliberately positioned on the surface of the metal.

Hybridization of target DNAs to such surface bound capture probes poses difficulties not seen, if both species are soluble. Steric effects result from the solid support itself and from too high of a probe density. Studies have shown that hybridization efficiency can be altered by the insertion of a linker moiety that raises the complementary region of the probe away from the surface (Schepinov et al., Nucleic Acid Res. 25:1155-1161 (1997); Day et al., Biochem J. 278:735-740 (1991)), the density at which probes are deposited (Peterson et al., Nucleic Acids Res. 29:5163-5168 (2001); Wilkins et al., Nucleic Acids Res. 27:1719-1729)), and probe conformation (Riccelli et al., Nucleic Acids Res. 29:996-1004 (2001)). Insertion of a linker moiety between the complementary region of a probe and its attachment point can increase hybridization efficiency and optimal hybridization efficiency has been reported for linkers between 30 and 60 atoms in length. Likewise, studies of probe density suggest that there is an optimum probe density, and that this density is less than the total saturation of the surface (Schepinov et al., Nucleic Acid Res. 25:1155-1161 (1997); Peterson et al., Nucleic Acids Res. 29:5163-5168 (2001); Steel et al., Anal. Chem. 70:4670-4677 (1998)). For example, Peterson et al. reported that hybridization efficiency decreased from 95% to 15% with probe densities of 2.0×10¹² molecules/cm² and 12.0×10¹² molecules/cm², respectively.

Quantitation of hybridization events often depends on the type of signal generated from the hybridization reaction. The most common analysis technique is fluorescent emission from several different types of dyes and fluorophores. However, quantitating samples in this manner usually requires a large amount of the signaling molecule to be present to generate enough emission to be quantitated accurately. More importantly, quantitation of fluorescence generally requires expensive analysis equipment for linear response. Furthermore, the hybridization reactions take up to two hours, which for many uses, such as detecting biological warfare agents, is simply too long. Therefore, a need exists for a system which can rapidly detect and quantitate biological material in samples.

The present invention is directed to achieving these objectives.

### SUMMARY OF THE INVENTION

One aspect of the present invention relates to a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card also has a second planar element parallel to and joined with the first planar element along a first planar interface. A fluid pathway is formed proximate to the first planar interface. The fluid pathway includes a detection reservoir into which at least part of the detection chip is received.

Another aspect of the present invention relates to a system for detecting a target molecule in a sample. This system includes a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card has a second planar element parallel to and joined with the first planar element along a first planar interface. A fluid pathway is formed proximate to the first planar interface. The fluid pathway includes a detection reservoir into which at least part of the detection chip is received. The detection card also has a first injection port through which a sample can be introduced into the detection card and electrical connectors coupled to the electrically separated conductors of the detection chip, such that the presence of a target molecule connecting the capture probes on the electrically separated conductors can be detected. The system further involves a support unit with respect to which the detection card can be positioned to carry out a procedure for detecting the target molecule in a sample. The support unit has an electrical coupler suitable for electrical communication with the electrical connectors of the detection card, so that the presence of the target molecule in the sample can be detected by the detection card and the support unit collectively.

A further aspect of the present invention relates to a method of detecting a target molecule. This method involves providing a detection system including a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card has a second planar element parallel to and joined with the first planar element along a first planar interface. A fluid pathway is formed proximate to the first planar interface. The fluid pathway includes a detection reservoir into which at least part of the detection chip is received. The detection card also has a first injection port through which a sample can be introduced into the detection reservoir and electrical connectors coupled to the electrically separated conductors of the detection chip, such that the presence of a target molecule connecting the capture probes on the electrically separated conductors can be detected. The system further involves a support unit with respect to which the detection card can be positioned to carry out a procedure for detecting the target molecule in a sample. The support unit has an electrical coupler suitable for electrical communication with the electrical connector of the detection card. The method further involves injecting a sample, potentially containing the target molecule, into the first injection port. The sample is then processed within the detection reservoir under conditions effective to permit any of the target molecule present in the sample to bind to the capture probes and thereby connect the capture probes. The presence of the target molecule is then detected by determining whether electricity is conducted between the electrically separated conductors.

In comparison to other detection systems which require the use of fluorescent or radioactive labels and a long reaction time, the present invention discloses a rapid and economical system for detecting target molecules in a sample. In particular, the disclosed system is amenable to being made portable for biological sample detection and identification, and is, thus, highly effective for many uses such as detecting biological warfare agents.

### BRIEF DESCRIPTION OF THE DRAWINGS

FIG. 1 is a top view of a detection card of the present invention. The dashed lines represent fluid pathways and a detection chip which reside underneath the top surface of the detection card.

FIG. 2 is an exploded perspective view of the detection card of FIG. 1 which has a first planar element, a second planar element, and a third planar element. Fluid pathways are formed by cut out portions of the second planar element and adjacent surfaces of the first and third planar elements.

FIG. 3 is a top view of an alternative embodiment of the detection card of the present invention. The dashed lines represent fluid pathways and a detection chip which reside underneath the surface of the detection card.

FIG. 4 is an exploded perspective view of the detection card of FIG. 3 which has a first planar element, a second planar element, and a third planar element. Fluid pathways are formed by cut out portions of the second planar element and adjacent surfaces of the first and third planar elements.

FIG. 5 is a top view of another embodiment of the detection card of the present invention. The dashed lines represent fluid pathways and a detection chip which reside underneath the surface of the detection card.

FIG. 6 is an exploded perspective view of the detection card of FIG. 5 which has a first planar element and a second planar element. Fluid pathways are formed by the indented portions of the second planar element and adjacent surfaces of the first planar element.

FIG. 7 is a top view of a further embodiment of the detection card of the present invention. The dashed lines represent fluid pathways and a detection chip which reside underneath the surface of the detection card.

FIG. 8 is an exploded perspective view of the detection card of FIG. 7 which has a first planar element and a second planar element. Fluid pathways are formed by the indented portions of the second planar element and adjacent surfaces of the first planar element.

FIG. 9 is a perspective view of a system for detection of a target nucleic acid molecule which includes a portable detection unit and a detection card which can be inserted into the portable unit.

FIG. 10 is a perspective view of a system for detection of a target nucleic acid molecule which includes a desk-top detection unit and a detection card which is inserted into the desk-top detection unit.

FIG. 11 is a schematic view of a system of FIG. 10 for detection of a target nucleic acid molecule from a sample.

FIG. 12A depicts a single test structure on a detection chip suitable to be positioned in the detection reservoir of the detection card of the present invention. Oligonucleotide probes are attached to electrical conductors in the form of spaced apart conductive fingers. FIG. 12B shows how a target nucleic acid molecule present in a sample is detected by the detection chip.

### DETAILED DESCRIPTION OF THE INVENTION

One aspect of the present invention relates to a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card also has a second planar element parallel to and joined with the first planar element along a first planar interface. A fluid pathway is formed proximate to the first planar interface. The fluid pathway includes a detection reservoir into which at least part of the detection chip is received.

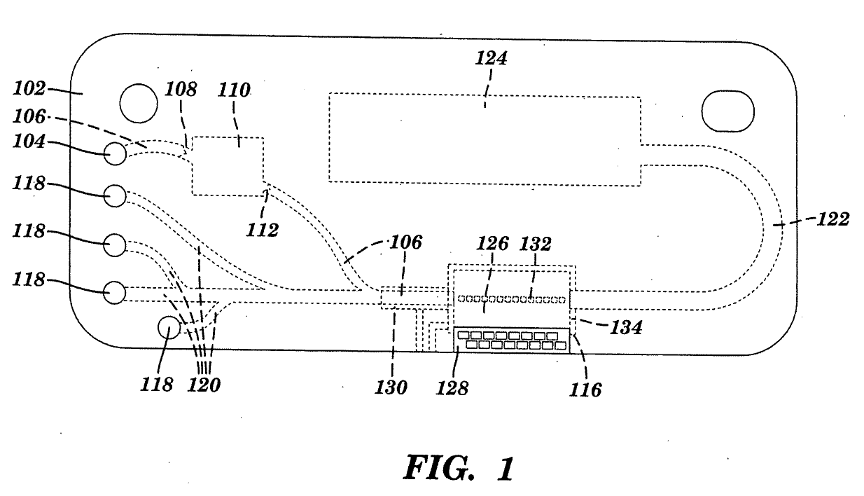

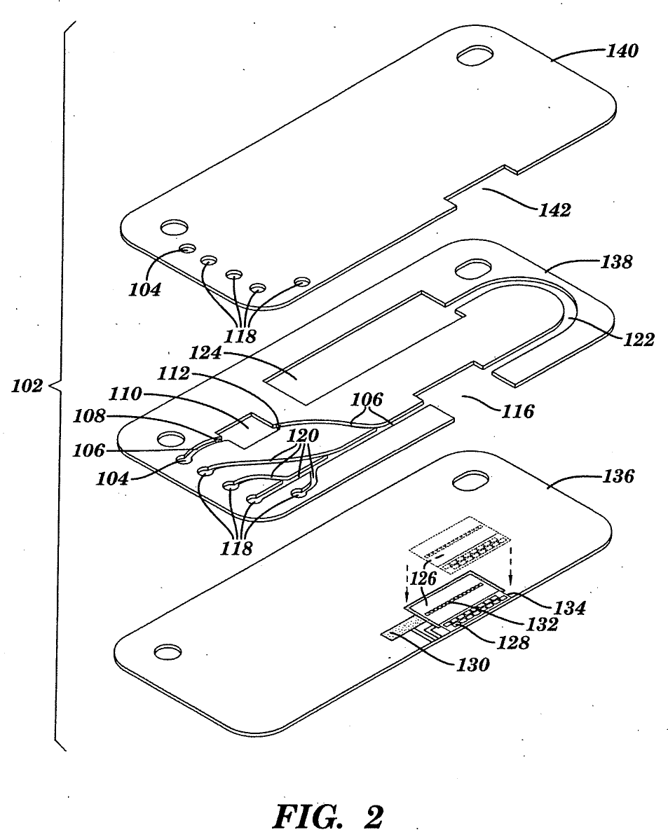

FIG. 1 is a top view of one embodiment of the detection card of the present invention. Detection card 102 has a fluid pathway in which a sample is introduced and analyzed for the presence of a target molecule. The sample is introduced into the fluid pathway of detection card 102 through first injection port 104. Channel 106 of the fluid pathway connects first injection port 104 to detection reservoir 116. Archival reservoir 110 is positioned in channel 106 between first injection port 104 and detection reservoir 116. Waste reservoir 124 is connected to detection reservoir 116 by channel 122. Reagents may also be introduced into the fluid pathway of detection card 102 through injection ports 118. Channels 120 connect injection ports 118 to channel 106 and, eventually, to detection reservoir 116.

The fluid pathway of detection card 102 also includes one-way valves that permit fluid to flow only in one direction. One-way valve 108 is positioned in channel 106 upstream of archival reservoir 110 to permit fluid to flow only from first injection port 104 to archival reservoir 110. One-way valve 112 is positioned in channel 106 between archival reservoir 110 and detection reservoir 1 16 to permit fluid to flow only from archival reservoir 110 to detection reservoir 116.

Detection card 102 also includes detection chip 126, heating element 134 and DNA concentrator 130. Detection chip 126 and on-chip sensor sites 132 reside at least partially within detection reservoir 116. A portion of detection chip 126 (i.e., card contacts 128) is exposed to the exterior of detection card 102. Heating element 134 is located near detection chip 126 and enables fluid temperatures in detection card 102, particularly detection reservoir 116, to be controlled by either heating or cooling the environment of detection reservoir 116. DNA concentrator 130 is located near the portion of channel 106 nearest detection reservoir 116. As described more fully in U.S. Provisional Patent Application Ser. No. 60/470,645, the DNA concentrator causes DNA in detection reservoir 116 to move closer to detection chip 126 by application of an electrode having a polarity which electrostatically attracts target molecules in a flowing fluid sample.

FIG. 2 is an exploded perspective view of the detection card of FIG. 1. Detection card 102 is comprised of three planar elements, including first planar element 136, second planar element 138, and third planar element 140, which, in use, are joined together. When second planar element 138 is joined to and positioned between first planar element 136 and third planar element 140, a fluid pathway is formed by the cut out pattern in second planar element 138 and adjacent surfaces of first planar element 136 and third planar element 140.

The cut out pattern in second planar element 138 includes first injection port 104, channel 106, detection reservoir 116, archival reservoir 110, channel 122, waste reservoir 124, injection ports 118, and channels 120. Third planar element 140 also has a cut out pattern, which creates sample injection port 104, injection ports 118, and cut out portion 142. Cut out portion 142 permits at least a portion of detection chip 126 (i.e., card contacts 128) to be exposed from detection card 102 when second planar element 138 is joined with and positioned between first planar element 136 and third planar element 140. First planar element 136 includes detection chip 126, DNA concentrator 130, and heating element 134, as described above.

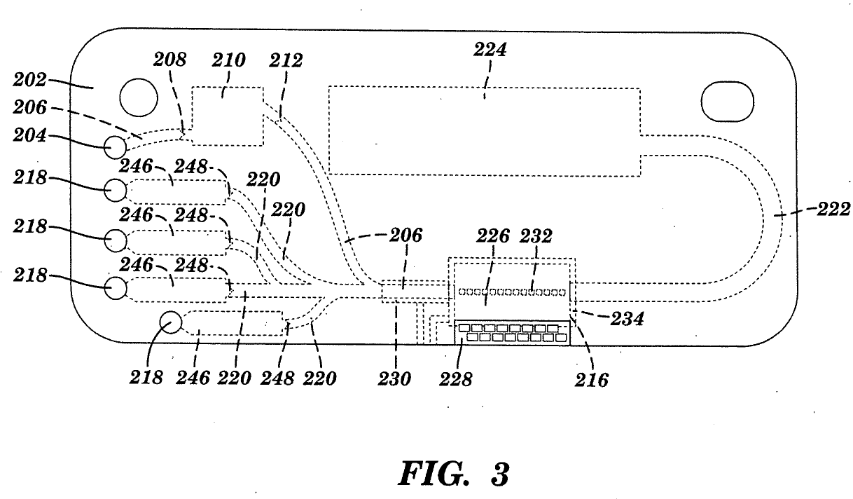

FIG. 3 is a top view of an alternative embodiment of the detection card of the present invention. Detection card 202 has a fluid pathway in which a sample is introduced and analyzed for the presence of a target molecule. The sample is introduced into the fluid pathway of detection card 202 through first injection port 204. Channel 206 connects first injection port 204 to detection reservoir 216. Archival reservoir 210 is positioned in channel 206 between first injection port 204 and detection reservoir 216. Waste reservoir 224 is connected to detection reservoir 216 by channel 222. Reagents may also be introduced into the fluid pathway of detection card 202 through injection ports 218. Reservoirs 246 are connected to injection ports 218 to receive and store reagents from injection ports 218. When ready for use, a material such as air, water, or oil can be forced into injection ports 218 of detection card 202 to move the reagents from reservoirs 246 into channels 220, and eventually into detection reservoir 216.

The fluid pathway of detection card 202 also includes one-way valves that permit fluid to flow only in one direction. One-way valve 208 is positioned in channel 206 upstream of archival reservoir 210 to permit fluid to flow only from first injection port 204 to archival reservoir 210. One-way valve 212 is positioned in channel 206 between archival reservoir 210 and detection reservoir 216 to permit fluid to flow only from archival reservoir 210 to detection reservoir 216. One-way valves 248 are positioned in channels 220 to permit fluid to flow only from reservoirs 246 to channel 206.

Detection card 202 also includes detection chip 226, heating element 234, and DNA concentrator 230, as described above. Detection chip 226 and on-chip sensor sites 232 reside at least partially within detection reservoir 216. A portion of detection chip 226 (i.e., card contacts 228) is exposed to the exterior of detection card 202. Heating element 234 is located near detection chip 226. DNA concentrator 230 is located near the portion of channel 206 nearest detection reservoir 216.

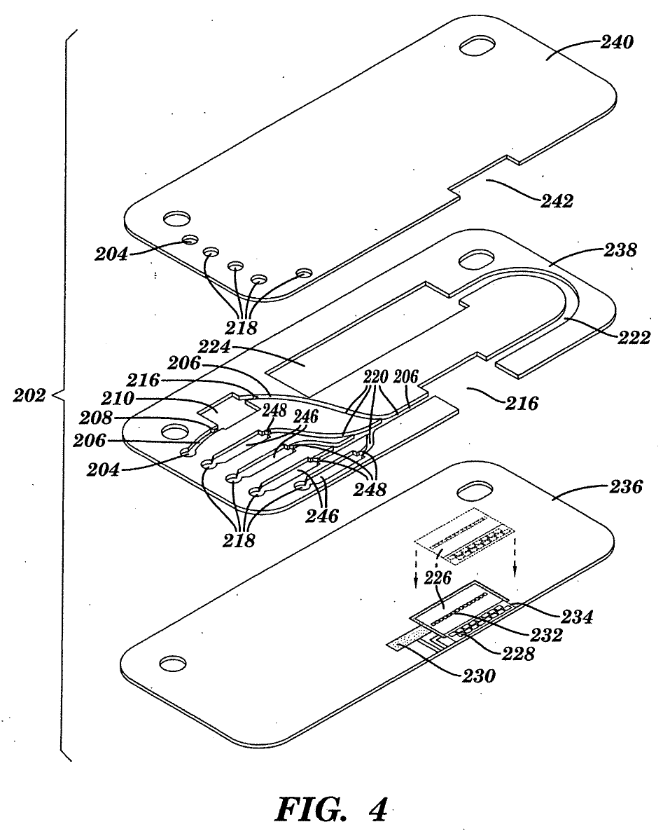

FIG. 4 is an exploded, perspective view of the detection card of FIG. 3. Detection card 202 is comprised of three planar elements, including first planar element 236, second planar element 238, and third planar element 240, which can be joined together. When second planar element 238 is joined with and positioned between first planar element 236 and third planar element 240, a fluid pathway is formed by the cut out pattern in second planar element 238 and adjacent surfaces of first planar element 236 and third planar element 240.

The cut out pattern in second planar element 238 includes first injection port 204, channel 206, detection reservoir 216, archival reservoir 210, channel 222, waste reservoir 224, injection ports 218, reservoirs 246, and channels 220. Third planar element 240 also has a cut out pattern, which creates sample injection port 204, injection ports 218, and cut out portion 242. Cut out portion 242 permits at least a portion of detection chip 226 (i.e., card contacts 228) to be exposed from detection card 202 when second planar element 238 is joined with and positioned between first planar element 236 and third planar element 240. First planar element 236 includes detection chip 226, DNA concentrator 230, and heating element 234.

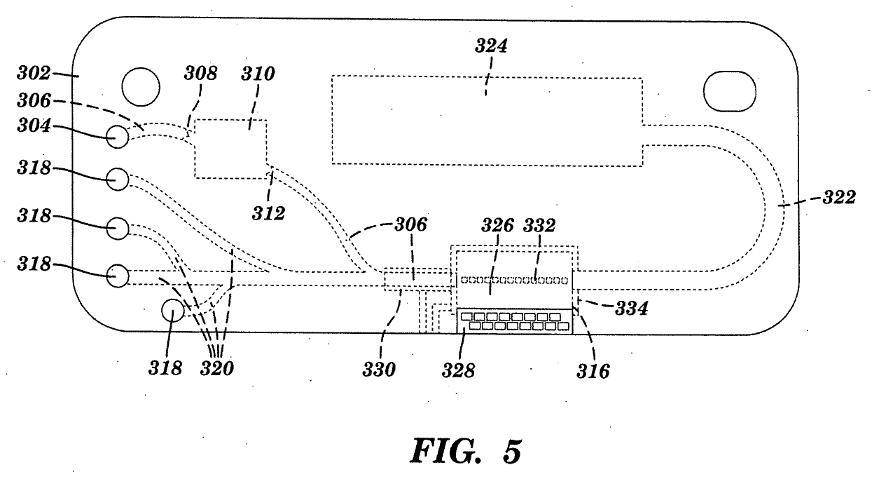

FIG. 5 is a top view of a further embodiment of the detection card of the present invention. Detection card 302 has a fluid pathway in which a sample is introduced and analyzed for the presence of a target molecule. The sample is introduced into the fluid pathway of detection card 302 through first injection port 304. Channel 306 of the fluid pathway connects first injection port 304 to detection reservoir 316. Archival reservoir 310 is positioned in channel 306 between first injection port 304 and detection reservoir 316. Waste reservoir 324 is connected to detection reservoir 316 by channel 322. Reagents may also be introduced into the fluid pathway of detection card 302 through injection ports 318. Channels 320 connect injection ports 318 to channel 306 and, eventually, to detection reservoir 316.

The fluid pathway of detection card 302 also includes one-way valves that permit fluid to flow only in one direction. One-way valve 308 is positioned in channel 306 upstream of archival reservoir 310 to permit fluid to flow only from first injection port 304 to archival reservoir 310. One-way valve 312 is positioned in channel 306 between archival reservoir 310 and detection reservoir 316 to permit fluid to flow only from archival reservoir 310 to detection reservoir 316.

Detection card 302 also includes detection chip 326, heating element 334, and DNA concentrator 330. Detection chip 326 and on-chip sensor sites 332 reside at least partially within detection reservoir 316. A portion of detection chip 326 (i.e., card contacts 328) is exposed to the exterior of detection card 302. Heating element 334 is located near detection chip 326. DNA concentrator 330 is located near the portion of channel 306 nearest detection reservoir 316.

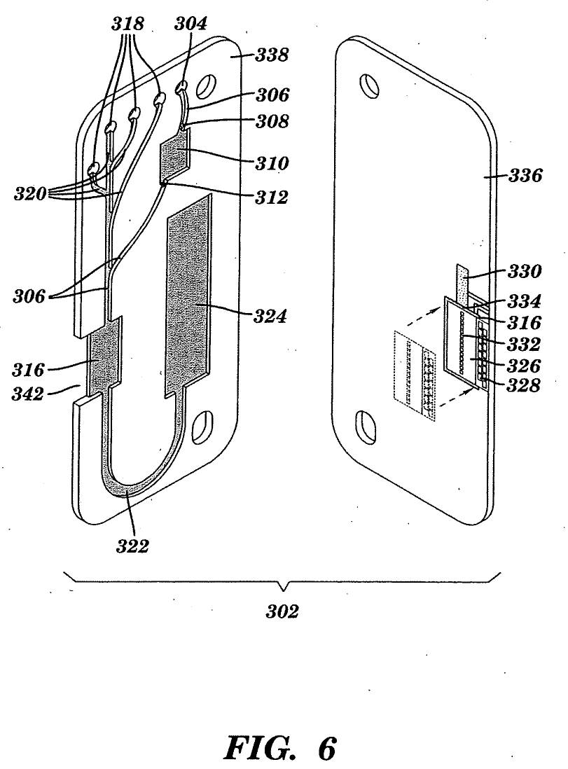

FIG. 6 illustrates an exploded perspective view of the detection card of FIG. 5. Detection card 302 is comprised of two planar elements—first planar element 336 and second planar element 338—which can be joined together. When first planar element 336 and second planar element 338 are joined together, a fluid pathway is formed by the indented pattern in second planar element 338 and the adjacent surface of first planar element 336.

The indented pattern of second planar element 338 includes channel 306, detection reservoir 316, archival reservoir 310, channel 322, waste reservoir 324, and channels 320. In addition to its indented pattern, second planar element 338 has cut out portions which make first injection port 304, injection ports 318, and cut out portion 342. Cut out portion 342 allows at least a portion of detection chip 326 (i.e., card contacts 328) to be exposed to the exterior surface of detection card 302 when first planar element 336 is joined with second planar element 338.

First planar element 336 of detection card 302 has detection chip 326, DNA concentrator 330, and heating element 334. When first planar element 336 is joined with second planar element 338, detection chip 326 and on-chip sensor sites 332 are at least partially received by detection reservoir 316 of the fluid pathway.

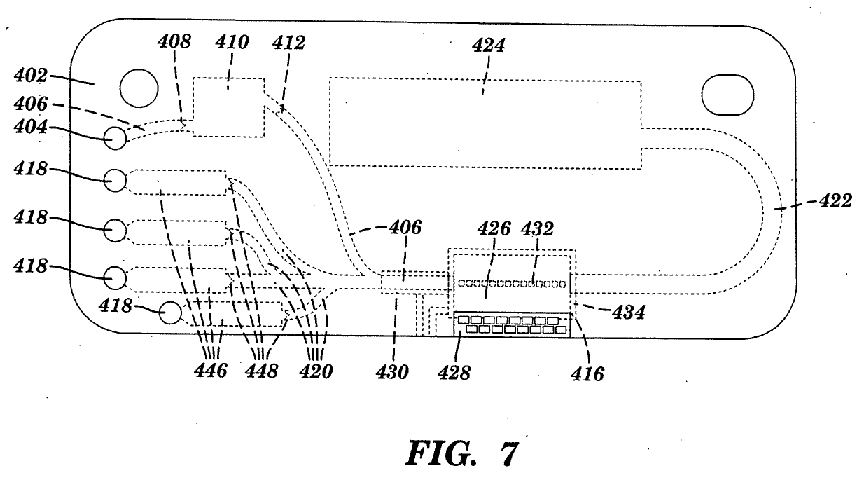

FIG. 7 is a top view of yet another embodiment of the detection card of the present invention. Detection card 402 has a fluid pathway in which a sample is introduced and analyzed for the presence of a target molecule. The sample is introduced into the fluid pathway of detection card 402 through first injection port 404. Channel 406 connects first injection port 404 to detection reservoir 416. Archival reservoir 410 is positioned in channel 406 between first injection port 404 and detection reservoir 416. Waste reservoir 424 is connected to detection reservoir 416 by channel 422. Reagents may also be introduced into the fluid pathway of detection card 402 through injection ports 418. Reservoirs 446 are connected to injection ports 418 to receive reagents from injection ports 418. Channels 420 connect reservoirs 446 to channel 406 and, eventually, to detection reservoir 416.

The fluid pathway of detection card 402 also includes one-way valves that permit fluid to flow only in one direction. One-way valve 408 is positioned in channel 406 upstream of archival reservoir 410 to permit fluid to flow only from first injection port 404 to archival reservoir 410. One-way valve 412 is positioned in channel 406 between archival reservoir 410 and detection reservoir 416 to permit fluid to flow only from archival reservoir 410 to detection reservoir 416. One-way valves 448 are positioned in channels 420 to permit fluid to flow only from reservoirs 446 to channel 406.

Detection card 402 also includes detection chip 426, heating element 434, and DNA concentrator 430. Detection chip 426 and on-chip sensor sites 432 reside at least partially within detection reservoir 416. A portion of detection chip 426 (i.e., card contacts 428) is exposed to the exterior of detection card 402. Heating element 434 is located near detection chip 426. DNA concentrator 430 is located near the portion of channel 406 nearest detection reservoir 416.

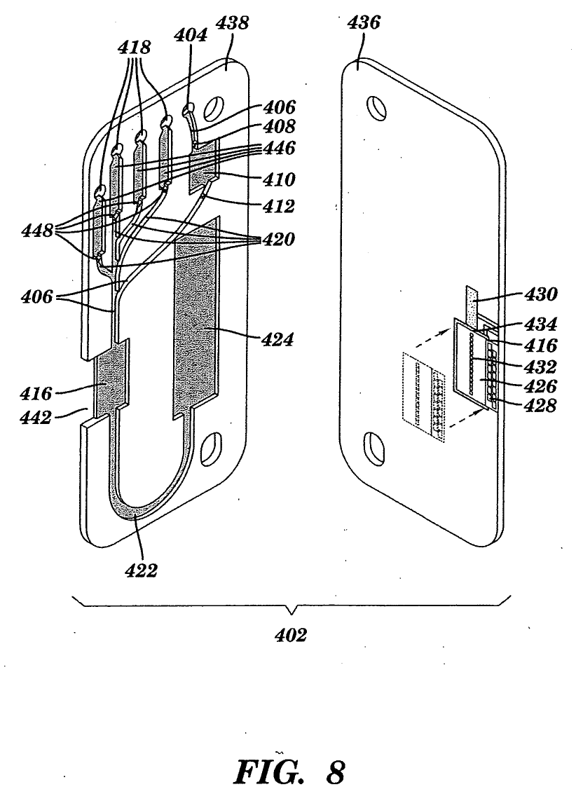

FIG. 8 is an exploded, perspective view of the detection card of FIG. 7. Detection card 402 is comprised of two planar elements—first planar element 436 and second planar element 438—which can be joined together. A fluid pathway is formed by the indented pattern in second planar element 438 and the adjacent surface of the first planar element 436.

The indented pattern of second planar element 438 includes channel 406, detection reservoir 416, archival reservoir 410, channel 422, waste reservoir 424, reservoirs 446, and channels 420. In addition to its indented pattern, second planar element 438 has cut out portions which form first injection port 404, injection ports 418, and cut out portion 442. Cut out portion 442 allows at least a portion of detection chip 426 (i.e., card contacts 428) to be exposed to the exterior surface of detection card 402 when first planar element 436 is bonded to second planar element 438.

First planar element 436 of detection card 402 has detection chip 426, DNA concentrator 430, and heating element 434. When first planar element 436 is joined with second planar element 438, detection chip 426 and on-chip sensor sites 432 are at least partially received by detection reservoir 416 of the fluid pathway.

The planar elements of the detection card of the present invention can be made from different types of materials such as plastics, metals, composites, glass, or a combination thereof for each different plane. Coating or adding performance agents such as additives to the planar elements may also be utilized for different performance expectations. The planar elements ;may contain printed or integrated circuits. Suitable plastics include, without limitation, thermoplastic resin systems, such as polyolefin and other hydrocarbon-based systems, and thermosetting resin systems, such as epoxies and urethane systems.

The planar elements can be joined together via many different methods. Such methods include, without limitation, adhesives, solvent binding, energy curable or activated systems, heat activated systems, ultrasonic welding or etching. Adhesive systems include, without limitation, heat activated adhesives, hot melt systems, pressure sensitive systems, energy-curable or activated adhesive systems, or liquid adhesive systems.

Another aspect of the present invention relates to a system for detecting a target molecule in a sample. This system includes a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card has a second planar element parallel to and joined with the first planar element along a first planar interface. A fluid pathway is formed proximate to the first planar interface. The fluid pathway includes a detection reservoir into which at least part of the detection chip is received. The detection card also has a first injection port through which a sample can be introduced into the detection card and electrical connectors coupled to the electrically separated conductors of the detection chip, such that the presence of a target molecule connecting the capture probes on the electrically separated conductors can be detected. The system further involves a support unit with respect to which the detection card can be positioned to carry out a procedure for detecting the target molecule in a sample. The support unit has an electrical coupler suitable for electrical communication with the electrical connectors of the detection card, so that the presence of the target molecule in the sample can be detected by the detection card and the support unit collectively.

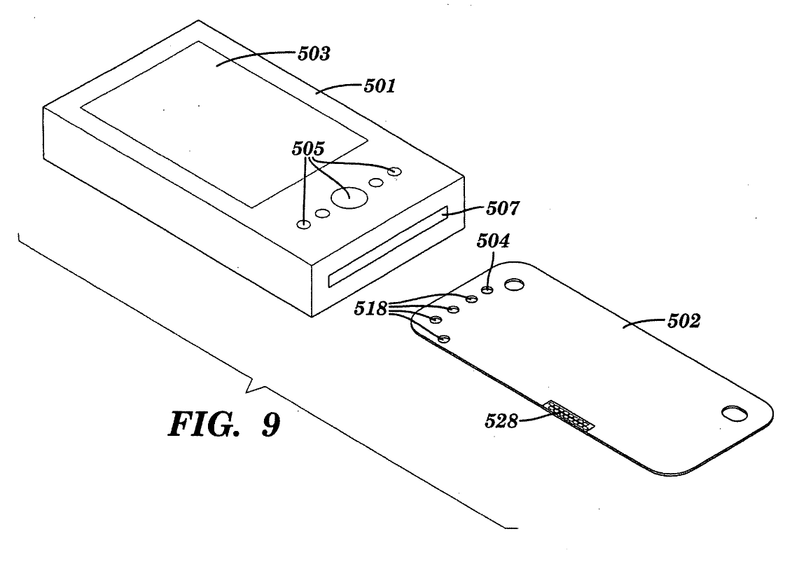

FIG. 9 shows a perspective view of a portable system for detecting a target molecule in a sample. This system includes a portable personal digital assistant (e.g., a Palm® unit, 3Com Corporation, Santa Clara, Calif.) and a detection card, as in FIG. 3 or FIG. 7, which is inserted into the portable personal digital assistant. In this embodiment, portable personal digital assistant 501 is provided. Portable personal digital assistant 501 is provided with visual display 503 and control buttons 505. Slot 507 is provided to receive detection card 502 having electrical connectors 528. Detection card 502 further contains first injection port 504 through which a sample solution can be introduced into detection card 502. Detection card 502 also contains fluid injection ports 518, through which reagents can be introduced into detection card 502.

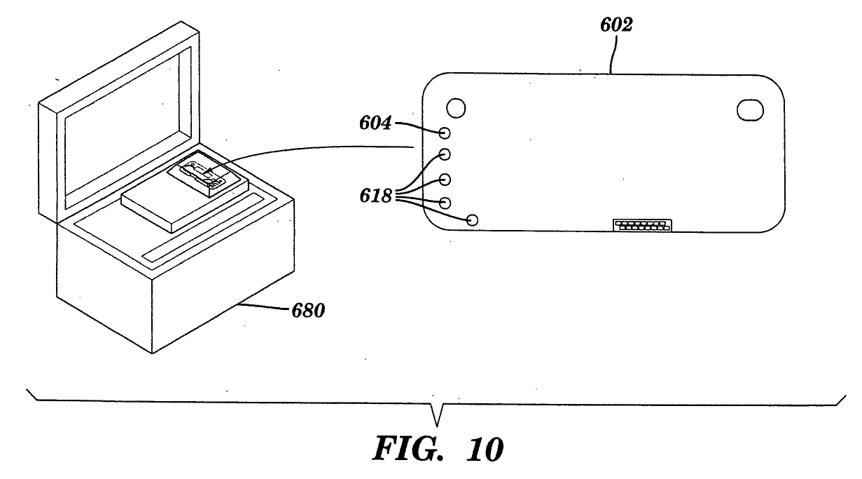

FIG. 10 shows a perspective view of a desk-top system for detecting a target molecule in a sample. This system includes desk-top detection unit 680 and detection card 602 which is inserted into the desk-top detection unit 680. Detection card 602 contains first injection port 604 through which a sample solution can be introduced into detection card 602 and also contains injection ports 618, through which reagents, stored in desk-top detection unit 680, can be introduced into detection card 602.

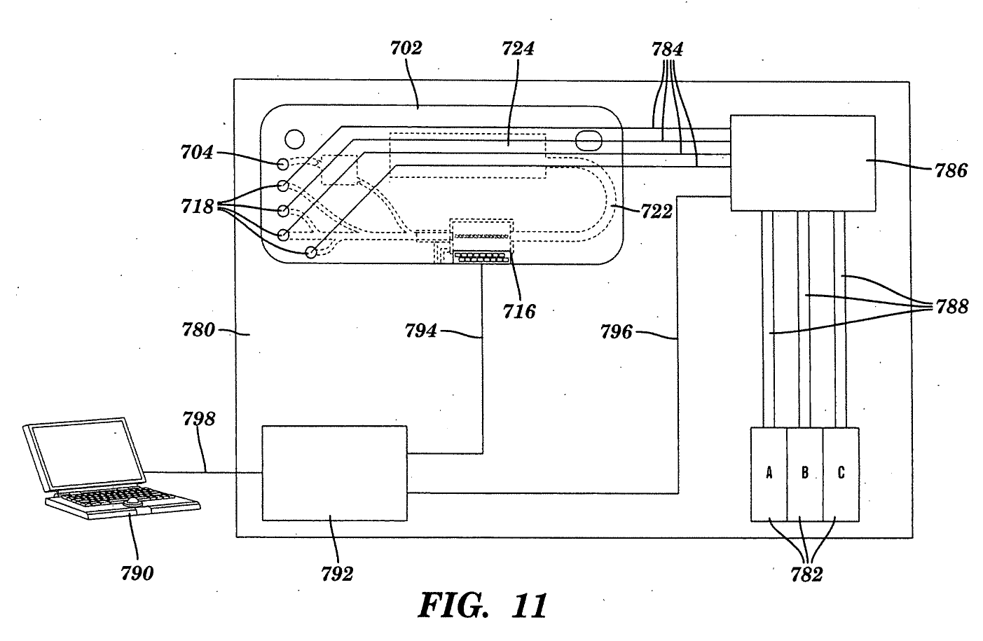

FIG. 11 shows a schematic view of a system for detecting a target molecule in a sample. The system may utilize a desk-top detection unit as described above. In this system, desk-top detection unit 780 contains containers 782A-C suitable for holding reagents and positioned to discharge the reagents into injection ports 718 through conduits 784. Containers 782A-C can, for example, carry a neutralizer, a buffer, a conductive ion solution, and an enhancer. The contents of these containers can be replenished. This is achieved by making containers 782A-C sealed and disposable or by making them refillable.

Pump 786 removes reagents from containers 782A-C, through conduits 788A-C, respectively, and discharges them through conduits 784 and fluid injection ports 718 into detection card 702. Instead of using single pump 786 to draw reagents from containers 782A-C, a separate pump can be provided for each of containers 782A-C so that their contents can be removed individually.

Alternatively, the necessary reagents may be held in reservoirs inside the detection card. For example, if detection card 202 illustrated in FIG. 3 was used in the system of the present invention, reagents may be stored in reservoirs 246. The pumps in the detection unit can force a material, such as air, water, or oil, into injection ports 218 of detection card 202 to force the reagents from reservoirs 246 into channels 220 and eventually into detection reservoir 216. The reagents are then changed with each detection card, which eliminates the buildup of salt precipitates in the detection unit.

Desk-top detection unit 780 is also provided with controller 792, which is in electrical communication with the electrical conductors of detection card 702 by means of electrical connector 794, to detect the presence of the target molecule in the sample. Controller 792 also operates pump 786 by way of electrical connector 796. Alternatively, separate controllers can be used for operating the pumps and the detection of target molecules. Digital coupling 798 permits controller 792 to communicate data to computer 790 which is external of desk-top detection unit 780.

Detection card 702 contains detection reservoir 716 which, as noted supra, receives reagents from within desk-top detection unit 780 by way of injection ports 718 and conduits 784. A sample to be analyzed is discharged into detection reservoir 716 through first injection port 704. As described more fully infra, the presence of a target molecule is detected in detection reservoir 716. Detection card 702 is further provided with waste reservoir 724 for collecting material discharged from detection reservoir 716 by way of fluid channel 722. Detection card 702 also contains electrical connector 794 coupled to the electrically separated conductors in detection reservoir 716 so that the presence of a target molecule in a sample can be detected.

The system of the present invention is used for the detection of nucleic acid sequences from a sample. This involves a sample collection method whereby bacteria, viruses, or other DNA containing species are collected and concentrated. The system also incorporates a sample preparation method that involves the liberation of the genetic components. After liberating the nucleic acid, the sample is injected into a detection card which includes a detection chip containing complementary nucleic acid probes for the target of interest. In this manner, the detection chip may contain multiple sets of probe molecules that each recognizes a single but different nucleic acid sequence. This process ultimately involves the detection of hybridization products.

In the collection phase, bacteria, viruses, or other DNA containing samples are collected and concentrated. A plurality of collection methods will be used depending on the type of sample to be analyzed. Liquid samples will be collected by placing a constant volume of the liquid into a lysis buffer. Airborne samples can be collected by passing air over a filter for a constant time. The filter will be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

After sample collection and lysis, cell debris can be removed by precipitation or filtration. Ideally, the sample will be concentrated by filtration, which is more rapid and does not require special reagents. Samples will be forced through filters that will allow only the cellular material to pass through, trapping whole organisms and broken cell debris.

In operation, the detection of a target molecule using a desk-top detection system, as shown in FIG. 11, can be carried out as follows. After lysis and clarification of the sample, the sample is introduced into detection card 702 through first injection port 704. The sample passes through channel 706 and into detection reservoir 716. Once the sample is introduced, detection card 702 is inserted into desk-top detection unit 780 so that fluid injection ports 718 are connected to conduits 784 and electrical connectors 728 are coupled to electrical connector 794. The sample is processed in detection reservoir 716 containing the capture probes and electrical conductors for a period of time sufficient for detection of a target nucleic acid molecule in the sample. Processing of the sample within detection reservoir 716 can involve neutralizing the sample, contacting the neutralized sample with a buffer, contacting the sample with metal ions, then contacting the sample with a metal deposition solution. Molecules that are not captured are expelled from detection reservoir 716 through channel 722 and into waste reservoir 724. Desk-top detection unit 780 can be programmed and the results can be seen on a visual display.

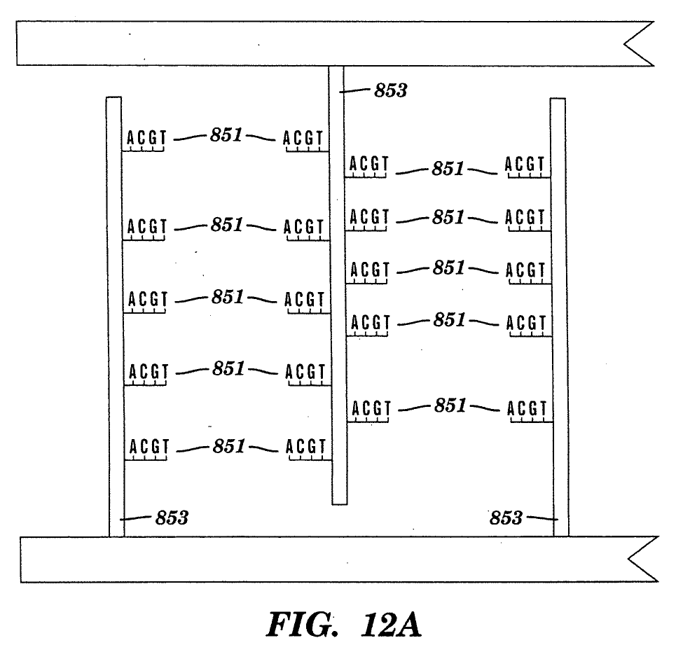

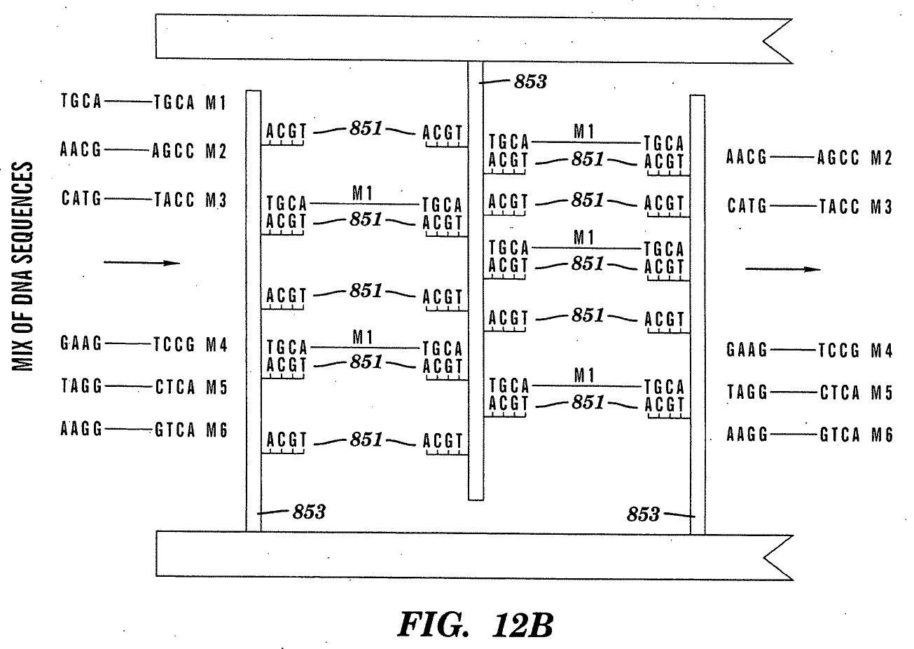

FIG. 12A depicts a single test structure of the detection chip of the present invention. According to FIG. 12A, oligonucleotide probes 851 attached to spaced apart conductive fingers 853 are physically located at a distance sufficient that they cannot come into contact with one another. A sample, containing a mixture of nucleic acid molecules (i.e., M1-M6) to be tested, is contacted with the fabricated device on which conductive fingers 853 are fixed, as shown in FIG. 12B. If a target nucleic acid molecule (i.e., M1) that is capable of binding to the two oligonucleotide probes is present in the sample, the target nucleic acid molecule will bind to the two probe molecules. If bound, the nucleic acid molecule can bridge the gap between the two electrodes and provide an electrical connection. Any unhybridized nucleic acid molecules (i.e., M2-M6) not captured by the probes is washed away.

Here, the electrical conductivity of nucleic acid molecules can be relied upon to transmit the electrical signal. Hans-Werner Fink and Christian Schoenenberger reported in Nature 398:407-410 (1999), which is hereby incorporated by reference in its entirety, that DNA conducts electricity like a semiconductor. This flow of current can be sufficient to construct a simple switch, which will indicate whether or not a target nucleic acid molecule is present within a sample. The presence of a target molecule can be detected as an “on” switch, while a set of probes not connected by a target molecule would be an “off” switch. The information can be processed by a digital computer which correlates the status of the switch with the presence of a particular target. The information can be quickly identified to the user as indicating the presence or absence of the biological material, organism, mutation, or other target of interest.

In a preferred embodiment of the present invention, after the target molecules have hybridized to sets of biological probes, the target molecule is contacted with metal ions under conditions effective to bind the metal ions on one or more sites of the target molecule. The target molecule with bound metal ions on one or more of its sites is then contacted with a metal under conditions effective to deposit metal on the target molecules hybridized to the probes as described in the U.S. patent application Ser. No. 10/763,597, which is hereby incorporated by reference in its entirety. Alternatively, metal particles may be mordanted on one or more sites of the nucleic acid molecule and metal deposited upon the mordanted nucleic acid molecule as described in U.S. Patent Application Ser. No. 60/533,342, which is hereby incorporated by reference in its entirety. The target nucleic acid molecule can then conduct electricity across the gap between the pair of probes. As described supra, this flow of current can be sufficient to construct a simple switch, which will indicate whether or not a target nucleic acid molecule is present within a sample.

The detection chip, on which conductive fingers 853 are fixed, is constructed on a support. Examples of useful support materials include, e.g., glass, quartz, and silicon as well as polymeric substrates, e.g. plastics. In the case of conductive or semi-conductive supports, it will generally be desirable to include an insulating layer on the support. However, any solid support which has a non-conductive surface may be used to construct the device. The support surface need not be flat. In fact, the support may be on the walls of a chamber in a chip.

The detection card described in this invention can be as simple as a device recognizing a single DNA sequence and hence a single organism, or as complex as recognizing multiple DNA sequences. Therefore, different types of detection cards can be constructed depending on the complexity of the application.

A further aspect of the present invention relates to a method of detecting a target molecule. This method involves providing a, detection system including a detection card having a first planar element with a detection chip. The detection chip has two or more electrically separated conductors fabricated on a substrate and capture probes attached to the conductors, such that a gap exists between the capture probes on the conductors. A sample, potentially containing a target molecule, can be analyzed for the presence of the target molecule by determining whether the conductors are electrically connected. The detection card has a second planar element parallel to and joined with the first planar element along a first planar interface. A fluid pathway is formed proximate to the first planar interface. The fluid pathway includes a detection reservoir into which at least part of the detection chip is received. The detection card also has a first injection port through which a sample can be introduced into the detection reservoir and electrical connectors coupled to the electrically separated conductors of the detection chip, such that the presence of a target molecule connecting the capture probes on the electrically separated conductors can be detected. The system further involves a support unit with respect to which the detection card can be positioned to carry out a procedure for detecting the target molecule in a sample. The support unit has an electrical coupler suitable for electrical communication with the electrical connector of the detection card. The method further involves injecting a sample, potentially containing the target molecule, into the first injection port. The sample is then processed within the detection reservoir under conditions effective to permit any of the target molecule present in the sample to bind to the capture probes and thereby connect the capture probes. The presence of the target molecule is then detected by determining whether electricity is conducted between the electrically separated conductors.

In carrying out the method of the present invention, a sample collection phase is initially carried out where bacteria, viruses, or other species are collected and concentrated. The target nucleic acid molecule whose sequence is to be determined is usually isolated from a tissue sample. If the target nucleic acid molecule is genomic, the sample may be from any tissue (except exclusively red blood cells). For example, whole blood, peripheral blood lymphocytes or peripheral blood mononuclear cells (“PBMC”), skin, hair, or semen are convenient sources of clinical samples. These sources are also suitable if the target is RNA. Blood and other body fluids are also a convenient source for isolating viral nucleic acids. If the target nucleic acid molecule is mRNA, the sample is obtained from a tissue in which the mRNA is expressed. If the target nucleic acid molecule in the sample is RNA, it may be reverse transcribed to DNA, but need not be converted to DNA in the present invention.

A plurality of collection methods can be used depending on the type of sample to be analyzed. Liquid samples can be collected by placing a constant volume of the liquid into a lysis buffer. Airborne samples can be collected by passing air over a filter for a constant time. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

When whole cells, viruses, or other tissue samples are being analyzed, it is typically necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations such as denaturation of contaminating (DNA binding) proteins, purification, filtration, and desalting.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea, to denature any contaminating and,potentially interfering proteins. Generally, where chemical extraction and/or denaturation methods are used, the appropriate reagents may be introduced into the fluid pathway of the detection card through the injection ports, the reagent reservoirs, or externally introduced.

Alternatively, physical methods may be used to extract the nucleic acids and denature DNA binding proteins. U.S. Pat. No. 5,304,487, which is hereby incorporated by reference in its entirety, discusses the use of physical protrusions within microchannels or sharp edged particles within a reservoir or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it is often desirable to separate the nucleic acids from other elements of the crude extract, e.g., denatured proteins, cell membrane particles, and the like. Removal of particulate matter is generally accomplished by filtration, flocculation, or the like. Ideally, the sample is concentrated by filtration, which is more rapid and does not require special reagents. A variety of filter types may be readily incorporated into the detection card of the present invention. Samples can be forced through filters that will allow only the cellular material to pass through, trapping whole organisms and broken cell debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional reservoir, or alternatively, the particular chromatographic media may be incorporated in a channel or fluid passage leading to a subsequent detection reservoir.

The probes are preferably selected to bind with the target such that they have approximately the same melting temperature. This can be done by varying the lengths of the hybridization region. A-T rich regions may have longer target sequences, whereas G-C rich regions would have shorter target sequences.

Hybridization assays on substrate-bound oligonucleotide arrays involve a hybridization step and a detection step. In the hybridization step, the sample potentially containing the target and an isostabilizing agent, denaturing agent, or renaturation accelerant is brought into contact with the probes of the array and incubated at a temperature and for a time appropriate to allow hybridization between the target and any complementary probes.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing-agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents.

Betaine (N,N,N,-trimethylglycine; (Rees et al., Biochem. 32:137-144 (1993)), which is hereby incorporated by reference in its entirety) can eliminate the base pair composition dependence of DNA thermal stability. Unlike tetramethylammonium chloride (“TMACI”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a composition that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M.

Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) A1 and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 μM to about 10 mM and, preferably, 1 μM to about 1 mM. The CTAB buffers work well at concentrations as low as 0.1 mM.

Addition of small amounts of ionic detergents (such as N-lauroyl-sarkosine) to the hybridization buffers can also be useful. LiCl is preferred to NaCl. Hybridization can be at 20°-65° C., usually 37° C. to 45° C. for probes of about 14 nucleotides. Additional examples of hybridization conditions are provided in several sources, including: Sambrook et al., Molecular Cloning: A Laboratory Manual, 2nd Ed., Cold Spring Harbor, N.Y. (1989); and Berger and Kimmel, “Guide to Molecular Cloning Techniques,” Methods in Enzymology, Volume 152, Academic Press, Inc., San Diego, Calif. (1987); Young and Davis, Proc. Natl. Acad. Sci. USA, 80:1194 (1983), which are hereby incorporated by reference in their entirety.

In addition to aqueous buffers, non-aqueous buffers may also be used. In particular, non-aqueous buffers which facilitate hybridization but have low electrical conductivity are preferred.

The sample and hybridization reagents are placed in contact with the detection chip and incubated in the detection reservoir of the detection card. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, hybridization is carried out at a temperature at or between five degrees below the melting temperature and the melting temperature. The target is incubated with the capture probes for a time sufficient to allow the desired level of hybridization between the target and any complementary capture probes. After incubation with the hybridization mixture, the electrically separated conductors are washed with the hybridization buffer, which also can include the hybridization optimizing agent. These agents can be included in the same range of amounts as for the hybridization step, or they can be eliminated altogether.

Details on how capture probes are attached to electrical conductors are set forth in U.S. patent application Ser. Nos. 10/288,657 and 10/763,597, which are hereby incorporated by reference in their entirety.

Various other methods exist for attaching the capture probes to the electrical conductors. For example, U.S. Pat. Nos. 5,861,242, 5,856,174, 5,856,101, and 5,837,832, which are hereby incorporated by reference in their entirety, disclose a method where light is shone through a mask to activate functional (for oligonucleotides, typically an —OH) groups protected with a photo-removable protecting group on a surface of a solid support. After light activation, a nucleoside building block, itself protected with a photo-removable protecting group (at the 5′-OH), is coupled to the activated areas of the support. The process can be repeated, using different masks or mask orientations and building blocks, to place probes on a substrate.

Alternatively, new methods for the combinatorial chemical synthesis of peptide, polycarbamate, and oligonucleotide arrays have recently been reported (see Fodor et al., Science 251:767-773 (1991); Cho et al., Science 261:1303-1305 (1993); and Southern et al., Genomics 13:1008-10017 (1992), which are hereby incorporated by reference in their entirety). These arrays (see Fodor et al., Nature 364:555-556 (1993), which is hereby incorporated by reference in its entirety) harbor specific chemical compounds at precise locations in a high-density, information rich format, and are a powerful tool for the study of biological recognition processes.

Preferably, the probes are attached to the leads through spatially directed oligonucleotide synthesis. Spatially directed oligonucleotide synthesis may be carried out by any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide which, itself, optionally has a protected active site if further nucleotide coupling is desired.

In one embodiment, the lead-bound oligonucleotides are synthesized at specific locations by light-directed oligonucleotide synthesis which is disclosed in U.S. Pat. No. 5,143,854, WO 92/10092, and WO 90/15070, which are hereby incorporated by reference in their entirety. In a basic strategy of this process, the surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′-O-phosphoramidite-activated deoxynucleoside (C—X) is presented to the surface. The selective photodeprotection and coupling cycles are repeated until the desired set of probes are obtained. Photolabile groups are then optionally removed, and the sequence is, thereafter, optionally capped. Side chain protective groups, if present, are also removed. Since photolithography is used, the process can be miniaturized to specifically target leads in high densities on the support.

The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the proteasd is removed or inactivated, the target nucleic acid molecule can be removed and the device is ready for use.

The capture probes can be formed from natural nucleotides, chemically modified nucleotides, or nucleotide analogs, as long as they have activated hydroxyl groups compatible with the linking chemistry. Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs, where the base moieties have been modified. In addition, analogs of oligomers may be polymers in which the sugar moiety has been modified or replaced by another suitable moiety, resulting in polymers which include, but are not limited to, polyvinyl backbones (Pitha et al., “Preparation and Properties of Poly (1-vinylcytosine),” Biochim. Biophys. Acta 204:381-8 (1970); Pitha et al., “Poly(1-vinyluracil): The Preparation and Interactions with Adenosine Derivatives,” Biochim. Biophys. Acta 204:39-48 (1970), which are hereby incorporated by reference in their entirety), morpholino backbones (Summerton, et al., “Morpholino Antisense Oligomers: Design, Preparation, and Properties,” Antisense Nucleic Acid Drug Dev. 7:187-9 (1997), which is hereby incorporated by reference in its entirety) and peptide nucleic acid (PNA) analogs (Stein et al., “A Specificity Comparison of Four Antisense Types: Morpholino, 2′-O-methyl RNA, DNA, and Phosphorothioate DNA,” J. Antisense Nucleic Acid Drug Dev. 7:151-7 (1997); Faruqi et al., “Peptide Nucleic Acid-Targeted Mutagenesis of a Chromosomal Gene in Mouse Cells,” Proc. Natl. Acad. Sci. USA 95:1398-403 (1998); Christensen et al., “Solid-Phase Synthesis of Peptide Nucleic Acids,” J. Pept. Sci. 1:175-83(1995); Nielsen et al., “Peptide Nucleic:Acid (PNA). A DNA Mimic with a Peptide Backbone,” Bioconjug. Chem. 5:3-7 (1994), which are hereby incorporated by reference in their entirety).

The capture probes can contain the following exemplary modifications: pendant moieties, such as, proteins (including, for example, nucleases, toxins, antibodies, signal peptides and poly-L-lysine); intercalators (e.g., acridine and psoralen), chelators (e.g., metals, radioactive metals, boron and oxidative metals), alkylators, and other modified linkages (e.g., alpha anomeric nucleic acids). Such analogs include various combinations of the above-mentioned modifications involving linkage groups and/or structural modifications of the sugar or base for the purpose of improving RNAseH-mediated destruction of the targeted RNA, binding affinity, nuclease resistance, and or target specificity.

The present invention can be used for numerous applications, such as detection of pathogens. For example, samples may be isolated from drinking water or food and rapidly screened for infectious organisms. The present invention may also be used for food and water testing. In recent times, there have been several large recalls of tainted meat products. The detection system of the present invention can be used for the in-process detection of pathogens in foods and the subsequent disposal of the contaminated materials. This could significantly improve food safety, prevent food borne illnesses and death, and avoid costly recalls. Capture probes that can identify common food borne pathogens, such as Salmonella and E. coli., could be designed for use within the food industry.

In yet another embodiment, the present invention can be used for real time detection of biological warfare agents. With the recent concerns of the use of biological weapons in a theater of war and in terrorist attacks, the device could be configured into a personal sensor for the combat soldier or into a remote sensor for advanced warnings of a biological threat. The devices which can be used to specifically identify the agent, can be coupled with a modem to send the information to another location. Mobile devices may also include a global positioning system to provide both location and pathogen information.

In yet another embodiment, the present invention may be used to identify an individual. A series of probes, of sufficient number to distinguish individuals with a high degree of reliability, are placed within the device. Various polymorphism sites are used. Preferentially, the device can determine the identity to a specificity of greater than one in one million, more preferred is a specificity of greater than one in one billion, even more preferred is a specificity of greater than one in ten billion.

### EXAMPLES

The following examples are provided to illustrate embodiments of the present invention but are by no means intended to limit its scope.

### Example 1

### Detection of Target Nucleic Acid Molecules in a Sample Containing Purified DNA

In a prophetic example, a 10 μl sample containing approximately 100 ng of purified DNA dissolved in hybridization buffer (100 mM NaPhosphate, pH 7.5, 0.1% SDS) with a defined length of 5.7 kilobases is injected into the archival reservoir. The nucleic acid denatures for approximately 1 minute before the reservoir is evacuated and the sample passed along to the detection reservoir. The nucleic acid sample resides in the detection reservoir over the test structures for 5 minutes at a temperature of 55 degrees. The sample is evacuated from the detection reservoir with a 10 sample volume wash with hybridization buffer. The nucleic acid sample is washed into the waste reservoir. A 10 sample volume wash with distilled and deionized water rinses out the reservoir and prepares the sensor for chemical coating. The metallization chemistry is then mixed on a card having electrically separated conductors and passed through the detection reservoir at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by passing a current (200 nA) through one of the two electrical test pads on each test structure and measuring the resistance between the two electrodes. Low resistance indicates the metallization process has fused two electrodes and is a positive result.

### Example 2

### Detection of Target Nucleic Acid Molecules in a Sample Containing Bacteria

In a prophetic example, a known quantity of bacteria are placed into lysis solution (Tris-CL, SDS) for 1 minute to break open bacteria. The cell debris is removed via filtration and the genomic DNA sheared by passing the solution through a point-sink shearing cartridge (65 μm diameter tubing). A 10 μl sample of the partially purified lysate in hybridization buffer (100 mM NaPhosphate, pH 7.5, 0.1% SDS) is injected into the archival reservoir. The nucleic acid denatures for approximately 1 minute before the reservoir is evacuated and the sample is passed along to the detection reservoir. The nucleic acid sample resides in the detection reservoir over the test structures for 5 minutes at a temperature of 55 degrees. The sample is evacuated from the detection reservoir with a 10 sample volume wash with hybridization buffer. The nucleic acid sample is washed into the waste reservoir. A 10 sample volume wash with distilled and deionized water rinses out the reservoir and prepares the sensor for chemical coating. The metallization chemistry is then mixed on a card having electrically separated conductors and passed through the detection reservoir at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by passing a current (200 nA) through one of the two electrical test pads on each test structure and measuring the resistance between the two electrodes. Low resistance indicates the metallization process has fused two electrodes and is a positive result.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A detection card comprising:
a first planar element comprising a detection chip, wherein the detection chip comprises:
two or more electrically separated conductors fabricated on a substrate, and
capture probes attached to the conductors such that a gap exists between the capture probes on the conductors, wherein a sample, potentially containing a target molecule, can be analyzed for the presence of the target molecule by determining whether the conductors are electrically connected; and
a second planar element parallel to and joined with the first planar element along a first planar interface, wherein a fluid pathway is formed proximate to the first planar interface, said fluid pathway including a detection reservoir into which at least part of the detection chip is received.

2. The detection card according to claim 1, wherein the two or more electrically separated conductors are in a form of spaced, apart conductive fingers.

3. The detection card according to claim 1, wherein a plurality of pairs of spaced apart conductive fingers are present on the detection chip.

4. The detection card according to claim 1, wherein the capture probes are oligonucleotides.

5. The detection card according to claim 1, wherein the capture probes are peptide nucleic acid analogs.

6. The detection card according to claim 1, wherein the fluid pathway further comprises:
a first injection port through which a sample can be introduced into the detection card.

7. The detection card according to claim 6, wherein the fluid pathway further comprises:
a first channel which connects the first injection port to the detection reservoir.

8. The detection card according to claim 7, wherein the fluid pathway further comprises:
a one-way valve in the first channel, permitting fluid only to flow from the first injection port to the detection reservoir.

9. The detection card according to claim 7, wherein the fluid pathway further comprises:
an archival reservoir positioned in the first channel between the first injection port and the detection reservoir.

10. The detection card according to claim 9 further comprising:
at least two one-way valves in the first channel upstream and downstream of the archival reservoir, permitting fluid only to flow from the first injection port to the detection reservoir.

11. The detection card according to claim 6, wherein the fluid pathway further comprises:
a waste reservoir for collecting material discharged from the detection reservoir and
a second channel establishing fluid communication between the detection reservoir and the waste reservoir.

12. The detection card according to claim 6, wherein the detection chip further comprises:
a heating element.

13. The detection card according to claim 6 further comprising:
a DNA concentrator upstream of the detection reservoir.

14. The detection card according to claim 6 further comprising:
electrical connectors extending through the detection card and coupled to the electrically separated conductors of the detection chip.

15. The detection card according to claim 6, wherein the fluid pathway further comprises:
a plurality of injection ports through which reagents can be introduced into the detection card.

16. The detection card according to claim 1, wherein the fluid pathway further comprises:
a plurality of reservoirs suitable for holding reagents and positioned to discharge the reagents into the detection reservoir.

17. The detection card according to claim 16, wherein the reservoirs comprise:
a reservoir carrying a neutralizer;
a reservoir carrying a buffer;
a reservoir carrying a conductive ion solution; and
a reservoir carrying an enhancer.

18. The detection card according to claim 16, wherein each of the plurality of reservoirs is connected to the detection reservoir by a plurality of channels.

19. The detection card according to claim 18 further comprising:
a one-way valve in each of the plurality of channels, permitting fluid only to flow from each of the plurality of reservoirs to the detection reservoir.

20. The detection card according to claim 1, wherein the second planar element has an indented pattern so that the fluid pathway is formed by the indented pattern in the second planar element and a surface of the first planar element which forms the first planar interface.

21. The detection card according to claim 1 further comprising:
a third planar element, wherein the second planar element has a cut-out pattern and is positioned between the first planar element and the third planar element with the second and third planar elements being joined together along a second planar interface, the fluid pathway being formed by the cut-out pattern in the second planar element and surfaces of the first and third planar elements which form the first and second planar interfaces respectively.

22. A system for detecting a target molecule in a sample, said system comprising:
a detection card comprising:
a first planar element comprising a detection chip, wherein the detection chip comprises:
two or more electrically separated conductors fabricated on a substrate, and
capture probes attached to the conductors such that a gap exists between the capture probes on the conductors, wherein a sample, potentially containing a target molecule, can be analyzed for the presence of the target molecule by determining whether the conductors are electrically connected; and
a second planar element parallel to and joined with the first planar element along a first planar interface, wherein a fluid pathway is formed proximate to the first planar interface, said fluid pathway including a detection reservoir into which at least part of the detection chip is received;
a first injection port through which a sample can be introduced into the detection card;
electrical connectors coupled to the electrically separated conductors of the detection chip, such that the presence of a target molecule connecting the capture probes on the electrically separated conductors can be detected; and
a support unit with respect to which the detection card can be positioned to carry out a procedure for detecting the target molecule in a sample, wherein said support unit has an electrical coupler suitable for electrical communication with the electrical connectors of the detection card, whereby the presence of the target molecule in the sample can be detected by the detection card and the support unit collectively.

23. The system according to claim 22, wherein the support unit has a slot into which the detection card is inserted.

24. The system according to claim 22, wherein the support unit is portable.

25. The system according to claim 24, wherein the support unit is a personal digital assistant.

26. The system according to claim 22, wherein the support unit is a desk-top unit.

27. The system according to claim 26, wherein the support unit further comprises:
a pump to remove reagents from the plurality of reservoirs and feed them into the detection reservoir through the plurality of injection ports.

28. The system according to claim 26, wherein the support unit further comprises:
a controller in communication with the electrical connectors to enable electrical communication between the electrical conductors of the detection card and the controller, whereby the presence of the target molecule in the sample can be detected by the detection card and support unit collectively.

29. The system according to claim 28, wherein the support unit further comprises:
a digital coupling to permit the controller to communicate data to a computer external of the support unit.

30. A method of detecting a target molecule comprising:
providing a detection system comprising:
a detection card comprising:
a first planar element comprising a detection chip, wherein the detection chip comprises:
two or more electrically separated conductors fabricated on a substrate, and
capture probes attached to the conductors such that a gap exists between the capture probes on the conductors, wherein a sample, potentially containing a target molecule, can be analyzed for the presence of the target molecule by determining whether the conductors are electrically connected; and
a second planar element parallel to and joined with the first planar element along a first planar interface, wherein a fluid pathway is formed proximate to the first planar interface, said fluid pathway including a detection reservoir into which at least part of the detection chip is received;
a first injection port through which a sample can be introduced into the detection reservoir;
electrical connectors coupled to the electrically separated conductors of the detection chip, such that the presence of a target molecule connecting the capture probes on the electrically separated conductors can be detected; and
a support unit with respect to which the detection card can be positioned to carry out a procedure for detecting the target molecule in a sample, wherein said support unit has an electrical coupler suitable for electrical communication with the electrical connector of the detection card;
injecting a sample, potentially containing the target molecule, into the first injection port;
processing the sample within the detection reservoir under conditions effective to permit any of the target molecule present in the sample to bind to the capture probes and thereby connect the capture probes; and
detecting the presence of the target molecule by determining whether electricity is conducted between the electrically separated conductors.

31. The method according to claim 30 further comprising:
collecting material discharged from the detection reservoir in a waste reservoir.

32. The method according to claim 30, wherein said processing comprises:
neutralizing the sample; and.
contacting the neutralized sample with a buffer, said method further comprising:
treating the sample with a conductive ion solution after said contacting with a buffer and
treating the sample with an enhancer after said treating with a conductive ion solution.

33. The method according to claim 30 further comprising:
coating any target molecules bound to the capture probes with a conductive ion material after said processing and before said detecting.

34. The method according to claim 30 further comprising:
inserting the detection card into a slot within the support unit.
